(19) United States
(12) Patent Application Publication
(10) Pub. No.: US 2004/0181051 A1
(43) Pub. Date: Sep. 16, 2004

(54) Process for the production of 3'-nucleoside prodrugs

(76) Inventors: Richard Storer, Folkestone (GB); Adel M. Moussa, Burlington, MA (US); Steven Mathieu, Salem, NH (US)

Correspondence Address: KING & SPALDING LLP, 191 PEACHTREE STREET, N.E., ATLANTA, GA, 30303-1763, US

(21) Appl. No.: 10/746,395

(22) Filed: Dec. 23, 2003

Related U.S. Application Data
(60) Provisional application No. 60/436,150, filed on Dec. 23, 2002.

(51) Int. Cl. C07H 19/067; C07H 19/073

(52) U.S. Cl. 536/27.1; 536/28.5; 536/28.4

(57) ABSTRACT

Provided is a single-step process for the selective 3′-acylation of a ribofuranosyl 2′ or 3′-branched nucleoside. These compounds are useful as antiviral agents, and in particular, can be used to treat Flaviviridae infections in a host in need thereof.

### CROSS-REFERENCE

This application claims priority to U.S. Provisional Application No. 60/436,150, filed on Dec. 23, 2002.

### FIELD OF THE INVENTION

This invention is a process for the preparation of 3′-acylated prodrugs of 2′-and 3′-branched ribofuranosyl nucleosides.

### BACKGROUND OF THE INVENTION

Historically, nucleoside prodrugs have usually been designed via acylation or other modification of the 5′-hydroxyl group of the nucleoside. Novirio Pharmaceuticals Limited (now Idenix Pharmaceuticals) discovered that the stability and bioavailability of certain 2′ and 3′ branched nucleosides (i.e., nucleosides that have four non-hydrogen substituents in the 2′ or 3′-positions) is enhanced by the administration of acylated forms of the nucleosides (See for example, WO 01/90121 (U.S. Ser. No. 09/864,078); WO 01/92282 (U.S. Ser. No. 09/863,816); PCT/IB03/03901 (U.S. Ser. No. 10/609,298); PCT/IB03/03246 (U.S. Ser. No. 10/608,907); and PCT/US03/20431 (U.S. Ser. No. 10/607,909)). Processes used for preparing these amino acid esters of nucleosides and nucleoside analogues began with appropriately branched β-D or β-L nucleosides that optionally could be protected by an appropriate protecting group such as, for example, a silyl group, and subsequently deprotected, by methods known to those skilled in the art (Zhang et al., Tetrahedron Letters, 1992, 33:1177-80; Greene et al., Protective Groups in Organic Synthesis, John Wiley & Sons, 2^{nd} Edition (1991); Kerr et al., J. Pharmaceutical Sciences, 1994, 83:582-6; Tang et al., J. Org. Chem., 1999, 64(3): 747-754; and Cavelier et al., Tetrahedron Letters, 1996, 37:5131-4). The optionally protected branched nucleoside was then coupled with a suitable acyl donor, such as an acyl chloride and/or an acyl anhydride or an activated acid, in an appropriate protic or aprotic solvent and at a suitable reaction temperature, to provide the 2′ or 3′ prodrug of the branched nucleoside, optionally in the presence of a suitable coupling agent (see Synthetic Communications, 1978, 8(5): 327-33; J. Am. Chem. Soc., 1999, 121(24):5661-5; Bryant et al., Antimicrob. Agents Chemother., 2001, 45, 229-235; Standring et al., Antiviral Chem. & Chemother., 2001, 12 (Suppl. 1), 119-129; Benzaria et al., Antiviral Res., 2001, 50, A79; Pierra et al., Antiviral Res., 2001, 50, A79; and Cretton-Scott et al., Antiviral Res., 2001, 50, A44). Examples of coupling reagents were any reagents that enable compounds or moieties to be linked to one another including, but not limited to, various carbodiimides, CDI, BOP and carbonyldiimidazole. For example, during the synthesis of a 3′-prodrug of a 2′-branched nucleoside, the nucleoside preferably was not protected, but was coupled directly to an alkanoic or amino acid residue via a carbodiimide-coupling reagent.

Matulic-Adamic et al. (U.S. Pat. No. 6,248,878) reported the synthesis of nucleoside analogues that comprise a ribofuranose ring with a phosphorus-containing group attached to the 3′-position via an oxygen atom and a substituted pyrimidine base. The phosphorus-containing group includes dithioates or phosphoramidites, or may be part of an oligonucleotide. These compounds are prodrugs because they are reacted further to provide final, desired nucleosides and nucleoside analogues. The compounds are synthesized in a multi-step process that couples, as starting materials, a ribofuranose having an hydroxy or acetoxy group at C-1 and benzoyl-protecting groups at C-2-, C-3 and C-5, and a 4-OSiMe₃ pyrimidine to produce an 1-(2,3,5-tri-O-benzoyl-ribofuranosyl)-pyrimidin-4-one; followed by the addition of ammonia in methanol to the product of the first reaction in order to remove the benzoyl protecting groups; then the reaction of DMT-Cl/Pyr with the unprotected product compound, which resulted in the addition of DMT to the 5′-O position of ribofuranose. The 5′-O-DMT substituted ribofuranose product was reacted with TBDMS-Cl, AgNO₃, and Pyr/THF. Standard phosphitylation was then carried out to produce the 3′-phosphorus-containing compound. Each of the syntheses presented included at least 4 to 7 steps.

In 1999, McCormick et al. described the preparation of the 3′-carbonate of guanosine, using an unprotected ribose as a starting material (McCormick et al., J. Am. Chem. Soc. 1999, 121(24):5661-5). McCormick was able to synthesize the compound by a sequential, stepwise introduction of the O- and N-glycosidic linkages, application of certain protecting groups, sulfonation and final deprotection. McCormick et al. reacted unprotected guanosine with BOC-anhydride, DMAP, Et₃N, and DMSO at room temperature for 4 hours to obtain directly the 3′-carbonate of guanosine.

Tang et al. disclosed a process for preparing phosphoramidite prodrugs of 2′-C-β-methyl-cytidine ribonucleosides (Tang et al., J. Org Chem., 1999, 64:747-754). Tang et al. reacted 1,2,3,5-tetra-O-benzoyl-2-C-methyl-β-D ribofuranose with persilylated 4-N-benzoylcytosine in the presence of the Lewis acid, SnCl₄, as a first step in the synthesis (Id. at 748, Scheme 1^{a}).

In view of the fact that 3′-acylated prodrugs of 2′- and 3′-branched nucleosides have importance as agents for the treatment of viral diseases, including flaviviruses, pestiviruses and notably hepatitis C, it would be advantageous to have an efficient process for selective addition of the acyl group to 3′-OH of the nucleoside.

Therefore, it is an object of the present invention to provide a process for the preparation of 3′-acylated derivatives of 2′ and 3′-branched nucleosides that can be used as a commercial scale manufacturing route.

It is another object to provide a synthesis of such compounds that minimizes the number of steps in the reaction.

It is another object to have a process that utilizes only non-toxic, inexpensive reagents, requires minimal special equipment or reaction conditions, and runs to completion within a short time.

It is yet another object of the present invention to provide an efficient process for preparing such compounds that provides a high yield of product.

### SUMMARY OF THE INVENTION

The present invention is a single-step process for the selective 3′-acylation of a ribofuranosyl 2′ or 3′-branched nucleoside. A ribofuranosyl nucleoside bears hydroxyl groups at the 2′ and 3′ positions. The process accomplishes the result of acylating the 3′-hydroxyl group but not the 2′-hydroxyl group.

In one embodiment, the process of the present invention utilizes inexpensive reagents, requires no special reaction conditions, and no special apparatus. For example, the process of the present invention can provide 3′-nucleoside prodrugs of 2′ and 3′-branched nucleosides in approximately 54% yield at about 98% purity.

An advantageous aspect of the present invention is that it requires only a single step. In one embodiment, the reaction takes only about 1 hour. In a particular embodiment of the present invention, the process can be used to selectively esterify the 3′-OH without protection of the other free hydroxyls, such as the 5′-hydroxyl. It is quite surprising that selective acylation of a compound with multiple hydroxyl groups can be accomplished so readily with this discovered process.

It was found that reacting a nucleoside with a protected organic acid in the presence of a coupling reagent (such as CDI), and a base (such as TEA), optionally in the presence of a base catalyst (such as DMAP), for example in a polar solvent (such as DMF and/or THF), results in the selective addition of the protected organic acid to the 3′-OH of the nucleoside, thereby forming a 3′-prodrug of the nucleoside. The process occurs in only a single step, and the time required for forming the prodrug product is significantly reduced from processes found in the prior art. In one embodiment of the present invention, the product yield is above 50%.

In one embodiment, the process of the present invention includes reacting a 2′ or 3′-branched ribofuranosyl nucleoside analogue with an acyl group, a lower alkanoyl, or derivative of an organic carboxylic acid to provide a 3′-nucleoside derivative prodrug. In another embodiment, the process of the present invention includes reacting the nucleoside analogue with a carboxylic acid derivative that has protecting groups on all functional groups except for the group of interest, to provide a nucleoside prodrug having an ester moiety. In yet another embodiment, the carboxylic acid derivative is a naturally-occurring or non-naturally-occurring amino acid.

As one illustration of the invention, the process of the present invention includes the single step of reacting a nucleoside with a free 3′-OH, such as 4-amino-1-(3,4-dihydroxy-5-hydroxymethyl-3-methyl-tetrahydro-furan-2-yl)-1H-pyrimidine-2-one, with BOC-valine/CDI and DMAP/TEA/DMF to form a 3′-O-valinoyl ester of the nucleoside, such as 2-tert-butoxycarbonylamino-3-methyl-butyric acid 5-(4-amino-2-oxo-2H-pyrimidin-1-yl)-4-hydroxy-2-hydroxymethyl-4-methyl-tetrahydro-furan-3-yl ester.

In one embodiment, BOC (t-butoxycarbonyl) is used as the protecting group for the amino acid. However, the process is not limited to the use of BOC and any nitrogen-protecting group such as, for example, an acyl or silyl group, may be used (see Greene et al., Protective Groups in Organic Synthesis, John Wiley & Sons, 3rd Edition (1999)). Also, CDI (carbonyl diimidazole) may be replaced by any coupling agent, such as a carbodiimide, used in the synthesis of dipolar polyamides and polypeptides.

The reaction can be carried out in any polar solvent. In one embodiment, either DMF or DMSO (dimethyl sulfoxide) is used. In an additional embodiment, THF (tetrahydrofuran) can be used as a co-solvent.

Similarly, any tertiary amine may replace TEA such as, for example, diisopropylethylamine and N-ethylmorpholine.

The nucleosides and nucleoside analogues are not limited to the compound exemplified, but embrace substituted and unsubstituted nucleoside bases, including purine bases, pyrimidine bases, pyrrolopyrimidines, triazolopyridines, imidazolopyridines, pyrazolopyrimidines, and the non-naturally occurring bases given below. The optionally substituted 5-membered rings may contain an O, S, or CH₂ group in place of the O atom of the furan. All stereoisomers and tautomeric forms of these nucleosides and nucleoside analogues are also included herein.

### BRIEF DESCRIPTION OF THE DRAWINGS

FIG. 1 is a non-limiting example of a process for direct esterification of the 3′-OH of a pyrimidine nucleoside of the present invention.

FIG. 2 is a non-limiting example of a process for direct esterification of the 3′-OH of a purine nucleoside of the present invention.

FIG. 3 is a prior art schematic of derivatization at the 3′-OH of guanosine.

### DETAILED DESCRIPTION OF THE INVENTION

The present invention provides an improved process for preparing a 3′-prodrug of a pharmaceutically active 2′ or 3′-branched ribofuranosyl nucleoside by selective acylation.

It was discovered unexpectedly that reacting a 2′ or 3′-branched nucleoside with a protected organic acid in the presence of a coupling reagent (such as CDI), base (such as TEA), optionally in the presence of a base catalyst (such as DMAP), and a polar solvent (such as THF and/or DMF) results in the addition of the protected organic acid selectively to the 3′-OH of the nucleoside, thereby forming a 3′-prodrug of the nucleoside. Since the process occurs in only a single step, the time required for forming the prodrug product is significantly reduced from processes found in the prior art. In one embodiment of the present invention, the product yield is above 50%.

Other unexpected advantage derived from this process include the low cost of reagents used. Another unexpected advantage derived from this process include the lack of extreme reaction conditions. Moreover, because the process does not require specialized equipment or apparatus, there is an additional cost savings to the user. Further, the process lends itself to easy scaleability for manufacturing purposes.

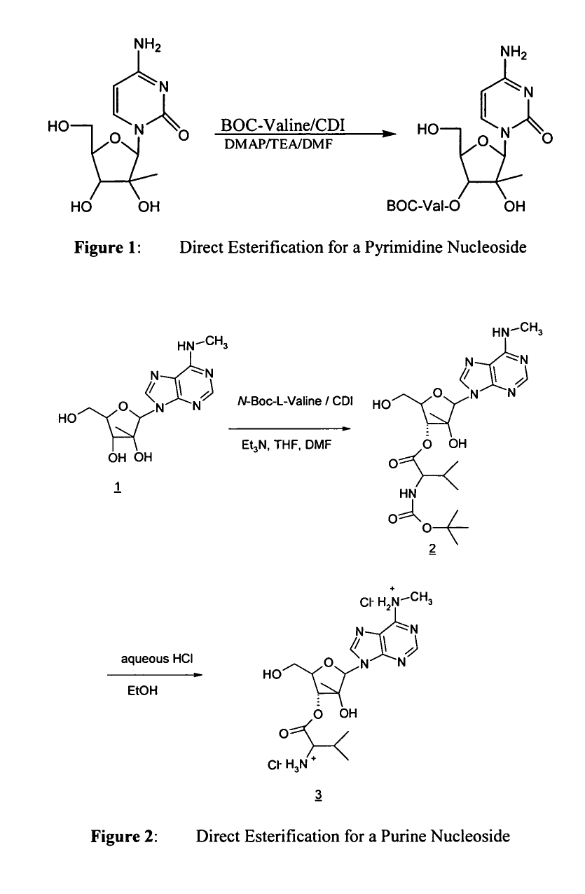

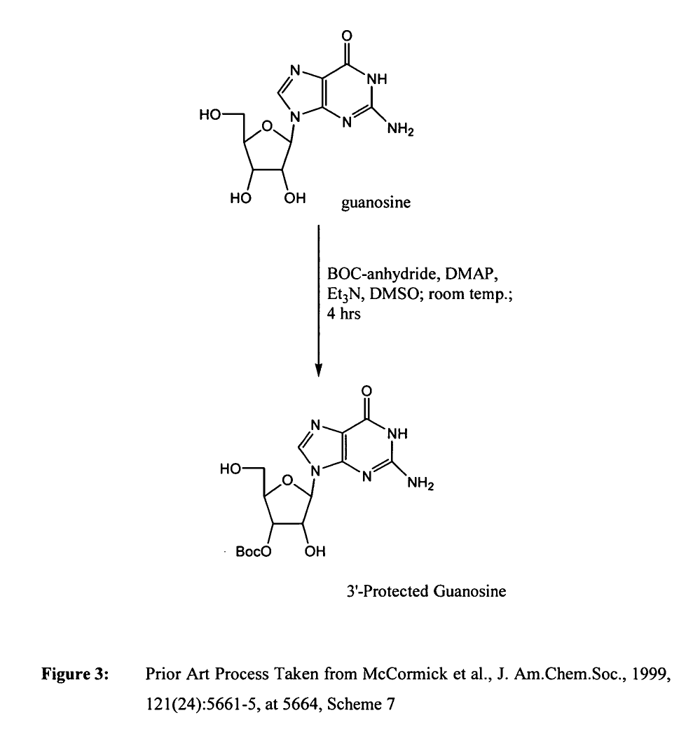

FIGS. 1 and 2 are schematics of the nonlimiting embodiments of the present invention. In the process described in FIG. 1, 4-amino-1-(3,4-dihydroxy-5-hydroxymethyl-3-methyl-tetrahydro-furanyl)-1H-pyrimidine-2-one is reacted with BOC-protected valine that is activated by CDI in THF or DMF. Of the solvents used in this process, THF can act as a co-solvent with DMF. TEA can be replaced with any tertiary amine such as, for example, diisopropylethylamine or N-ethylmorpholine, and DMF may be replaced by other polar solvents such as, for example, DMSO (dimethylsulfoxide) or NMP (N-methylpyrrolidinone). This exemplary process has a reaction time of approximately 1 hour.

Nucleosides and nucleoside analogues that can be derivatized using this process are not limited to the compounds exemplified, but can include, for example, substituted and unsubstituted nucleoside bases, including purine bases, pyrimidine bases, pyrrolopyrimidines, triazolopyridines, imidazolopyridines, pyrazolopyrimidines, and the non-naturally occurring bases described below. The optionally substituted 5-membered ring may contain an O, S, or CH₂ group in place of the O atom of the furan. All stereoisomers and tautomeric forms of these nucleosides and nucleoside analogues are also included herein.

Detailed Description of the Process Steps

The nucleoside with a free or reactive 3′-OH (or —SH) can be purchased or can be prepared by any published or unpublished means including standard reduction, oxidation, substitution and/or coupling techniques. In the main embodiment, the nucleoside is a 2′ or 3′-branched nucleoside. In an alternative embodiment, the nucleoside with a free 3′-OH (or —SH) is a 2′-deoxynucleoside such as 2′-deoxycytidine or 2′-deoxythymidine, which can be purchased or can be prepared by any published or unpublished means including standard reduction and coupling techniques. In another embodiment of the present invention, the nucleoside with a free 3′-OH is a 2′-branched nucleoside such as 4-amino-1-(3,4-dihydroxy-5-hydroxymethyl-3-methyl-tetrahydro-furanyl)-1H-pyrimidine-2-one (β-D-2′-C-methyl-cytidine) or 9-(2′-C-methyl-β-D-ribofuranosyl)-6-N-methyl-adenine, which can be purchased or can be prepared by any published or unpublished means including standard oxidation, substitution and coupling techniques. In yet another embodiment of the present invention, the nucleoside with a free 3′-OH is a 3′-branched nucleoside, which can be purchased or can be prepared by any published or unpublished means including standard oxidation, substitution and coupling techniques. Another example of a starting material is β-D-2′-C-methyl-N-methyl-purine.

The optionally protected organic acid can be purchased or can be prepared by any published or unpublished means. In one embodiment of the invention, the optionally protected organic acid is an optionally protected amino acid, such as a Boc-protected amino acid, preferably a Boc-protected L-valine. The free amino group of the amino acid can be selectively protected with a suitable protecting group, preferably with an acyl group, such as —(C═O)-aralkyl, —(C═O)-alkyl or —(C═O)-aryl, preferably BOC (butoxycarbonyl), by methods well known to those skilled in the art, as taught in Greene, et al., Protective Groups in Organic Synthesis, John Wiley and Sons, Second Edition, 1991. The process of the present invention is not limited to the use of BOC as a protecting group. Other protecting groups such as, for example, substituted or unsubstituted silyl groups; substituted or unsubstituted ether groups like C—O-aralkyl, C—O-alkyl, or C—O-aryl; aliphatic groups such as acyl or acetyl groups having an alkyl moiety that is straight-chained or branched; and any such groups that would not adversely affect the materials, reagents and conditions of the present invention as known to those of skill in the art and as taught by Greene et al., Protective Groups in Organic Synthesis, John Wiley and Sons, 2^{nd} Edition (1991), may be used.

The 3′-selectively acylated nucleoside can be prepared by reaction of the optionally protected organic acid with the nucleoside with a free 3′-OH (or —SH) in the presence of a coupling reagent and base(s). Suitable coupling reagents include EDC (1-[3-(dimethylamino)-propyl]-3-ethyl-carbodiimide hydrochloride); also referred to as DEC), CDI (carbonyldiimidazole), BOP reagent (benzotriazol-1-yloxy-tris(dimethylamino)-phosphonium hexafluorophosphate), Mitsunobu reagents (e.g., diisopropyl azodicarboxylate and diethyl azodicarboxylate) with triphenylphosphine, other carbodiimides or similar coupling reagents as known to those skilled in the art, though preferably CDI. Suitable bases include TEA (triethylamine) diisopropylethylamine, N-ethylmorpholine, any tertiary aliphatic amine or other suitable amine, or a combination thereof, preferably TEA, which can be optionally used in combination with a base catalyst, such as DMAP.

The optionally protected organic acid and/or coupling reagent can be reacted with the nucleoside at any molar ratio that allows the reaction to proceed at an acceptable rate without excessive side products, such as with a slight molar excess, for example at a about a 1.0 to about 1.5 molar excess of coupling reagent, preferably about 1.1 to about 1.25 molar excess, and/or about a 1.0 to about 1.5 molar excess of optionally protected organic acid, preferably about 1.1 to about 1.25 molar excess, to nucleoside. In one embodiment, the base(s) can be reacted using an excess amount. If the base(s) are used in combination with a base catalyst, such as DMAP, then in one embodiment, the base catalyst, such as DMAP is used in catalytic amounts, for example about 0.1:1 molar ratio to the nucleoside.

In one embodiment, the reagents can be added simultaneously or sequentially over a suitable period and temperature to allow the reaction to proceed at an acceptable rate without excessive side products.

In one embodiment, the optionally protected organic acid is stirred with the coupling reagent prior to addition of the nucleoside and/or base(s). For example, the optionally protected organic acid, such as an optionally protected amino acid, for example Boc-L-valine, can be stirred with the coupling agent, such as CDI. This reaction can be accomplished at any temperature that allows the reaction to proceed at an acceptable rate without promoting decomposition or excessive side products. The preferred conditions are at from about room temperature to about 25° C., for about an hour to an hour and a half, and then heated to about 40-50° C. for about 20-30 minutes, preferably under inert conditions, for example under argon gas. This activated optionally protected organic acid can be prepared in any solvent that is suitable for the temperature and the solubility of the reagents. Solvents can consist of any polar aprotic solvent including, but not limiting to, acetone, ethyl acetate, dithianes, THF, dioxane, acetonitrile, dichloromethane, dichloroethane, diethyl ether, pyridine, dimethylformamide (DMF), DME, dimethylsulfoxide (DMSO), dimethylacetamide, or any combination thereof, though preferably THF.

In one embodiment, the nucleoside with a free 3′-OH (or —SH), such as 2′-deoxycytidine, 2′-deoxythymidine, 4-amino-1-(3,4-dihydroxy-5-hydroxymethyl-3-methyl-tetrahydro-furanyl)-1H-pyrimidine-2-one or 9-(2′-C-methyl-β-D-ribofuranosyl)-6-N-methyl-adenine or 9-(2′-C-methyl-β-D-ribofuranosyl)-6-N-methyl-purine, is stirred with base(s), optionally in the presence of a base catalyst, such as DMAP, prior to addition to the optionally protected organic acid and/or coupling reagent. For example, the nucleoside with a free 3′-OH (or —SH) can be stirred with the base(s), optionally in the presence of a base catalyst, such as DMAP. This reaction can be accomplished at any temperature that allows the reaction to proceed at an acceptable rate without promoting decomposition or excessive side products. The preferred conditions are temperatures that allow for the nucleoside to be completely solublized in the solvent, for example at about 95-100° C. for about 20-30 minutes, preferably under inert conditions, for example under argon gas. This activated nucleoside can be prepared in any solvent that is suitable for the temperature and the solubility of the reagents. Solvents can consist of any polar aprotic solvent including, but not limiting to, acetone, ethyl acetate, dithianes, THF, dioxane, acetonitrile, dichloromethane, dichloroethane, diethyl ether, pyridine, dimethylformamide (DMF), DME, dimethylsulfoxide (DMSO), dimethylacetamide, or any combination thereof, though preferably DMF.

In one embodiment of the invention, the activated organic acid (with coupling reagent) is then stirred with the activated nucleoside (with base(s), optionally in the presence of a base catalyst, such as DMAP). The two solutions can be added all at once or incrementally over a suitable period and temperature to allow the reaction to proceed at an acceptable rate without excessive side products. In one embodiment of the invention, the activated optionally protected organic acid is added incrementally over about a 2 hour period. In an alternate embodiment of the invention, the activated optionally protected organic acid is added quickly, for example, over about a 2 minute period. This reaction can be accomplished at any temperature that allows the reaction to proceed at an acceptable rate without promoting decomposition or excessive side products. In one example, the reaction solution is at about 80-100° C. during the addition of the activated optionally protected organic acid, and then from about 80-90° C. for about one hour, and then cooled to about room temperature, preferably under inert conditions, for example under argon gas. In one embodiment, the temperature is not reduced to below 80° C. during the addition of the activated optionally protected organic acid.

The reaction can be allowed to proceed until a substantial amount of the nucleoside is consumed, during which time reaction progression can be monitored, for example by taking aliquots periodically for TLC or HPLC analysis.

Once the reaction has proceeded to the desired point, some of the more volatile solvents (e.g. THF) and base(s) (e.g. TEA) optionally can removed by any means known in the art, for example under vacuum at a temperature of about 30° C., prior to quenching with an acid.

In a preferred embodiment, the process of the present invention is accomplished in one closed system, without any intermediary purification steps, i.e. a “one-pot” synthesis.

The reaction solution then can be neutralized if desired with an acid, such as acetic acid, to a pH of about 7.5 to about 7.75.

Any solvent not previously removed (e.g. DMF) can then be removed by any means known in the art, for example under vacuum at a temperature of about 35° C.

The product can be extracted from the crude solution by any means known in the art, including standard extraction and crystallization techniques. For example, the crude solution can be mixed with an organic solvent, such as ethyl acetate, methylene chloride, or tert-butyl methyl ether (MTBE), and water. The two layers can be separated, and again the aqueous layer can be extracted with an organic solvent, such as ethyl acetate, methylene chloride, or tert-butyl methyl ether (MTBE). The process of adding organic solvent and separating the resulting aqueous layer can be repeated as many times as necessary. The organic layers can be combined and optionally washed with an aqueous saturated brine solution. The resulting organic layer then can be extracted with an aqueous acidic solution, for example an aqueous solution of malonic acid. The organic layer can be checked, for example by TLC (thin layer chromatography), to be certain that all the desired product has been removed from the organic layer. In one embodiment, the acidic aqueous extracts then can be combined, cooled, for example in an ice bath, to about 0-10° C., and neutralized to a pH of about 7.4, for example using a base such as triethylamine, such that the desired product can precipitate from the solution. In an alternate embodiment, the acidic aqueous extracts then can be combined, cooled, for example in an ice bath, to about 0-10° C., neutralized to a pH of about 7.4, for example using a base such as triethylamine, and the aqueous layer is extracted with an organic solvent, such as MTBE. The process of adding organic solvent and separating the resulting aqueous layer can be repeated as many times as necessary. The combined organic layers can be dried over a drying agent, such as magnesium sulfate or sodium sulphate, and subsequently concentrated, for example under vacuum.

If desired, the 3′-selectively esterified nucleoside can be made into a pharmaceutically acceptable salt using any means known in the art. Pharmaceutically acceptable salts include those derived from pharmaceutically acceptable inorganic or organic acids and bases. Non-limiting examples of suitable salts include those derived from inorganic acids such as, hydrochloric acid, hydrobromic acid, sulfuric acid, phosphoric acid, nitric acid, bicarbonic acid, carbonic acid and the like, and salts formed with organic acids such as amino acid residue, acetic acid, oxalic acid, tartaric acid, succinic acid, malic acid, malonic acid, ascorbic acid, citric acid, benzoic acid, tannic acid, palmoic acid, alginic acid, polyglutamic acid, tosic acid, methanesulfonic acid, naphthalenesulfonic acid, naphthalenedisulfonic acid, α-ketoglutaric acid, α-glycerophosphoric acid and polygalacturonic acid. Suitable salts include those derived from alkali metals such as lithium, potassium and sodium, alkaline earth metals such as calcium and magnesium, among numerous other acids well known in the pharmaceutical art. Other suitable salts include those derived from other metal cations such as zinc, bismuth, barium, aluminum, copper, and the like, or with a cation formed from an amine, such as ammonia, N,N-dibenzylethylene-diamine, D-glucosamine, tetraethylammonium, or ethylene-diamine. Further, suitable salts include those derived from a combinations of acids and bases, for example, a zinc tannate salt or the like. Therefore, in one embodiment of the present invention, the 3′-selectively esterified at the 3′-position nucleoside can be reacted with a pharmaceutically acceptable inorganic or organic acid, such as HCl, in a solvent, such as a polar protic solvent, for example EtOH, to provide a pharmaceutically acceptable salt, such as a hydrochloride salt, as a final product.

Illustrative Embodiment

In one embodiment, a process for selectively esterifying the 3′ hydroxyl position of a 2′-branched ribofuranosyl nucleoside is provided comprising:

a) heating a first solution of a 2′ branched ribofuranosyl nucleoside in an organic solvent at temperature and for a time sufficient to dissolve the nucleoside;

b) adding a tertiary amine and a base catalyst to the first solution; and

c) adding a second solution, comprising a protected amino acid and a carbodiimide coupling reagent in an organic solvent, to the first solution.

The first solution is optionally heated to at least 80° C. for at least 20 minutes. Optionally in step c) the first solution is maintained at a temperature of at least 80° C., and the second solution is added over a time period of at least one hour. Optionally, the process further comprises heating the combined first and second solutions at a temperature of at least 80° C. for at least about one half hour. The organic solvent in the first solution is, e.g., a polar aprotic solvent, such as DMF. The organic solvent in the second solution is, e.g., a polar aprotic solvent, such as, THF or DMF. The process of claim 64, further comprising neutralizing the product solution with an acid. The tertiary amine is e.g. triethylamine and the base catalyst is e.g. DMAP. The protected amino acid can be a protected L-valinoyl amino acid.

In one embodiment, a solution of N-(tert-butoxycarbonyl)-L-valine in anhydrous THF or DMF is added to CDI and stirred at 25° C. under argon gas for about 1.5 hours, and then at 40-50° C. for 20 minutes. Into a separate flask outfitted with an argon gas line is added 4-amino-1-(3,4-dihydroxy-5-hydroxymethyl-3-methyl-tetrahydro-furanyl)-1H-pyrimidine-2-one in an amount just slightly less than a 1:1 molar ratio compared with that of the N-(tert-butoxycarbonyl)-L-valine dissolved in DMF, to which TEA and DMAP are added. The 4-amino-1-(2,3-dihydroxy-5-hydroxymethyl-2-methyl-tetrahydro-furanyl)-1H-pyrimidine-2-one then is heated to an external temperature of 100° C. for about 20 minutes or until the pyrimidine-2-one derivative compound is completely in solution, after which TEA and DMAP are added. This mixture is heated for about 20 minutes at approximately 97° C. (external temperature), and then the THF solution containing N-(tert-butoxycarbonyl)-L-valine is added slowly over an approximate 2 hour period at a temperature not lower than 82° C. (internal temperature). Next, the reaction mixture is heated at about 82° C. for approximately 1 hour, after which it is cooled to room temperature. Once cooled, the TEA and THF are removed under vacuum at a temperature of about 30° C.

Next the solution is neutralized with acetic acid to a pH of about 7.69, and DMF is removed under vacuum at a temperature of about 35° C. The solution is chased with ethyl acetate, and the crude product is stirred with ethyl acetate and water. The two layers are separated, and again the aqueous layer is extracted with ethyl acetate. Next the two organic layers are combined and washed with an aqueous saturated brine solution; the resulting organic layer is extracted with an aqueous solution of malonic acid. The organic layer is checked by TLC (thin layer chromatography) to be certain that all the desired product has been removed.

The acidic aqueous extracts then are combined, cooled in an ice bath, and neutralized with TEA to a pH of 7.4. At this pH the solids precipitate from the solution. Ethyl acetate is added to the aqueous layer, and white solids are collected and dried by vacuum filtration to provide the prodrug product.

Suitable Nucleosides for the Esterification Process

Any nucleoside or nucleoside analog with a free 3′-OH (or —SH) can be used in the processes of the present invention. Therefore, the present invention includes processes for the preparation of a 3′-prodrug of a nucleoside or nucleoside analog comprising reacting in a single closed system (i.e “one-pot” system) (a) a nucleoside or nucleoside analog with a free 3′-OH (or —SH); (b) an optionally protected organic acid, such as an optionally protected amino acid, for example Boc-L-valine; (c) a coupling reagent; and (d) a base, optionally in the presence of a base catalyst. In an additional embodiment, the pharmaceutically acceptable salt of 3′-prodrug of the nucleoside or nucleoside analog is desired. The pharmaceutically acceptable salt of 3′-prodrug of the nucleoside or nucleoside analog can be made using any means known in the art, including for example further adding an acidic salt to the 3′-prodrug of the nucleoside or nucleoside analog.

In one embodiment, base is a purine base. In another embodiment, base is a pyrimidine base. In yet another embodiment, base is a pyrrolopyrimidine. In yet another embodiment, base is a triazolopyridine, an imidazolopyridine, or a pyrazolopyrimidine. In a particular embodiment, the base is a pyrimidine base selected from the group consisting of thymine, cytosine, 5-fluorocytosine, 5-methylcytosine, 6-aza-pyrimidine, including 6-azacytosine, 2- and/or 4-mercaptopyrmidine, uracil, 5-halouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, and C⁵-aminopyrimidine.

In a particular sub-embodiment, the base is a selected from the group consisting of:

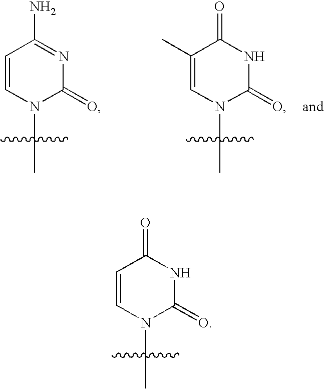

In another particular embodiment, the base is a purine base selected from the group consisting of N⁶-alkylpurines (including N-methyl purine), N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-thioalkyl purine, N²-alkylpurines, N²-alkyl-6-thiopurines, N²-alkylpurines, N²-alkyl-6-thiopurines, 5-azacytidinyl, guanine, adenine, hypoxanthine, 2,6-diaminopurine, and 6-chloropurine.

In another particular sub-embodiment, the base is a selected from the group consisting of:

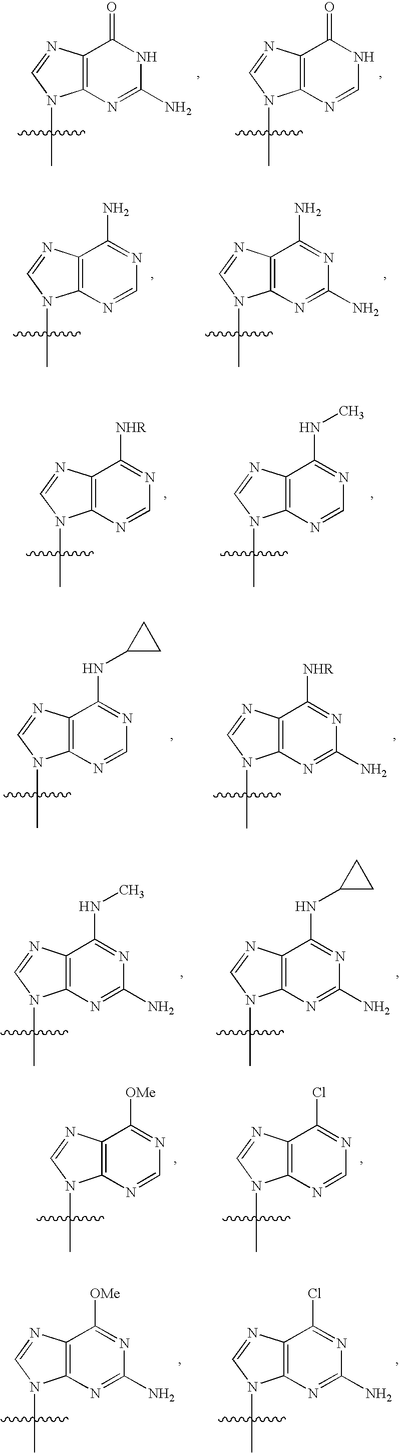

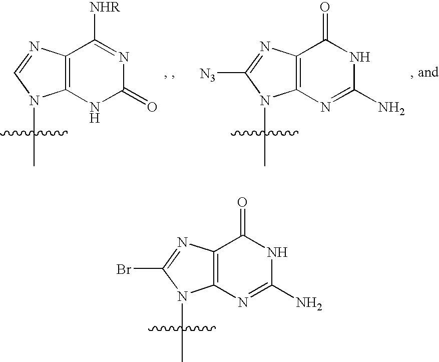

In one particularly preferred sub-embodiment of the invention, the 2′-C-methyl branched nucleoside to be selectively esterified at the 3′-position is

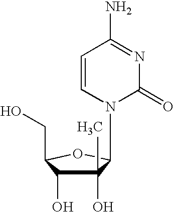

without protection of the free 2′- and/or 5′-OH.

In another particularly preferred sub-embodiment of the invention, the 2′-C-methyl branched nucleoside to be selectively esterified at the 3′-position is

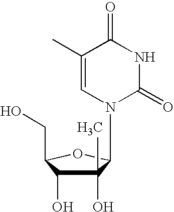

without protection of the free 2′- and/or 5′-OH.

In yet another particularly preferred sub-embodiment of the invention, the 2′-C-methyl branched nucleoside to be selectively esterified at the 3′-position is

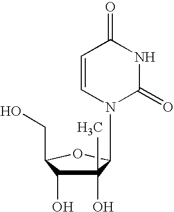

without protection of the free 2′- and/or 5′-OH.

In yet another particularly preferred sub-embodiment of the invention, the 2′-C-methyl branched nucleoside to be selectively esterified at the 3′-position is

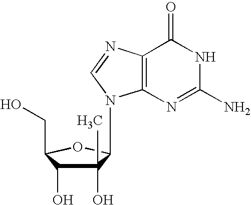

without protection of the free 2′- and/or 5′-OH.

In yet another particularly preferred sub-embodiment of the invention, the 2′-C-methyl branched nucleoside to be selectively esterified at the 3′-position is

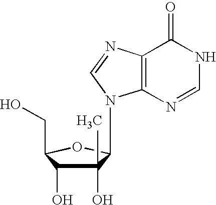

without protection of the free 2′- and/or 5′-OH.

In yet another particularly preferred sub-embodiment of the invention, the 2′-C-methyl branched nucleoside to be selectively esterified at the 3′-position is

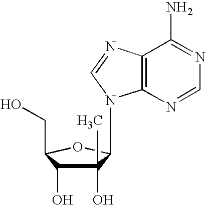

without protection of the free 2′- and/or 5′-OH.

In yet another particularly preferred sub-embodiment of the invention, the 2′-C-methyl branched nucleoside to be selectively esterified at the 3′-position is

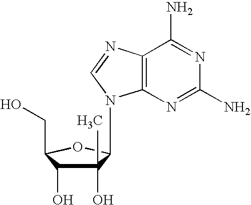

without protection of the free 2′- and/or 5′-OH.

In yet another particularly preferred sub-embodiment of the invention, the 2′-C-methyl branched nucleoside to be selectively esterified at the 3′-position is

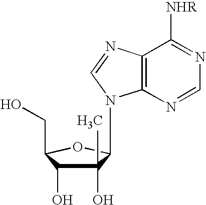

without protection of the free 2′- and/or 5′-OH;

wherein R is methyl, ethyl, propyl, isopropyl, cyclopropyl, butyl, isobutyl, t-butyl, pentyl, cyclopentyl, isopentyl, or neopentyl.

In yet another particularly preferred sub-embodiment of the invention, the 2′-C-methyl branched nucleoside to be selectively esterified at the 3′-position is

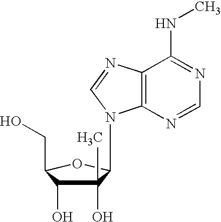

without protection of the free 2′- and/or 5′-OH.

In yet another particularly preferred sub-embodiment of the invention, the 2′-C-methyl branched nucleoside to be selectively esterified at the 3′-position is

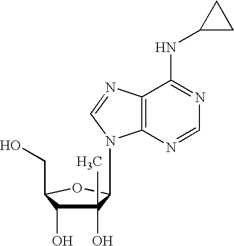

without protection of the free 2′- and/or 5′-OH.

In yet another particularly preferred sub-embodiment of the invention, the 2′-C-methyl branched nucleoside to be selectively esterified at the 3′-position is

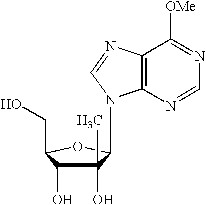

without protection of the free 2′- and/or 5′-OH.

In yet another particularly preferred sub-embodiment of the invention, the 2′-C-methyl branched nucleoside to be selectively esterified at the 3′-position is

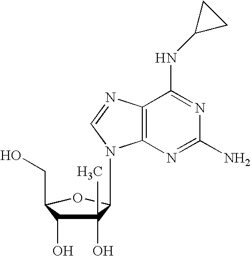

without protection of the free 2′- and/or 5′-OH.

In yet another particularly preferred sub-embodiment of the invention, the 2′-C-methyl branched nucleoside to be selectively esterified at the 3′-position is

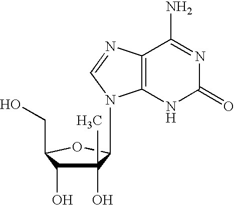

without protection of the free 2′- and/or 5′-OH.

In yet another particularly preferred sub-embodiment of the invention, the 2′-C-methyl branched nucleoside to be selectively esterified at the 3′-position is

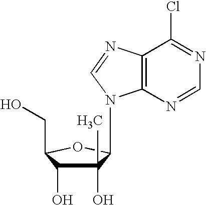

without protection of the free 2′- and/or 5′-OH.

In yet another particularly preferred sub-embodiment of the invention, the 2′-C-methyl branched nucleoside to be selectively esterified at the 3′-position is

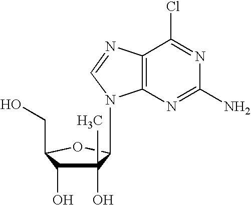

without protection of the free 2′- and/or 5′-OH.

In yet another particularly preferred sub-embodiment of the invention, the 2′-C-methyl branched nucleoside to be selectively esterified at the 3′-position is

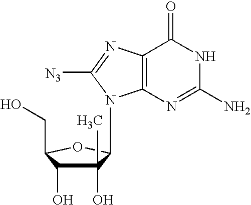

without protection of the free 2′- and/or 5′-OH.

In yet another particularly preferred sub-embodiment of the invention, the 2′-C-methyl branched nucleoside to be selectively esterified at the 3′-position is

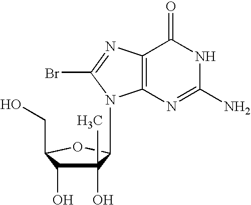

without protection of the free 2′- and/or 5′-OH.

In yet another particularly preferred sub-embodiment of the invention the 2′-C-methyl branched nucleoside to be selectively esterified at the 3′-position is

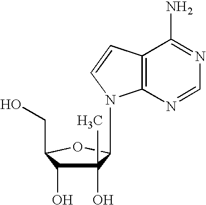

without protection of the free 2′- and/or 5′-OH.

In yet another particularly preferred sub-embodiment of the invention, the 2′-C-methyl branched nucleoside to be selectively esterified at the 3′-position is

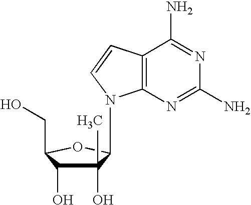

without protection of the free 2′- and/or 5′-OH.

In yet another particularly preferred sub-embodiment of the invention, the 2′-C-methyl branched nucleoside to be selectively esterified at the 3′-position is

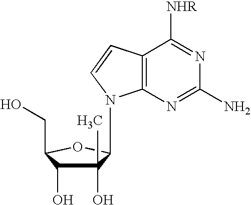

without protection of the free 2′- and/or 5′-OH.

wherein R is methyl, ethyl, propyl, isopropyl, cyclopropyl, butyl, isobutyl, t-butyl, pentyl, cyclopentyl, isopentyl, or neopentyl.

In yet another particularly preferred sub-embodiment of the invention, the 2′-C-methyl branched nucleoside to be selectively esterified at the 3′-position is

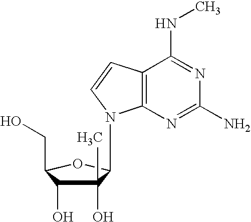

without protection of the free 2′- and/or 5′-OH.

In yet another particularly preferred sub-embodiment of the invention, the 2′-C-methyl branched nucleoside to be selectively esterified at the 3′-position is

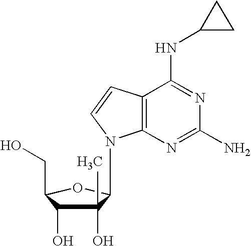

without protection of the free 2′- and/or 5′-OH.

In yet another particularly preferred sub-embodiment of the invention, the 2′-C-methyl branched nucleoside to be selectively esterified at the 3′-position is

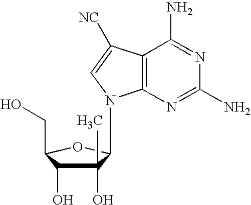

without protection of the free 2′- and/or 5′-OH.

In yet another particularly preferred sub-embodiment of the invention, the 2′-C-methyl branched nucleoside to be selectively esterified at the 3′-position is

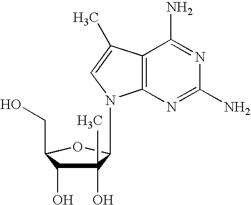

without protection of the free 2′- and/or 5′-OH.

In yet another particularly preferred sub-embodiment of the invention, the 2′-C-methyl branched nucleoside to be selectively esterified at the 3′-position is

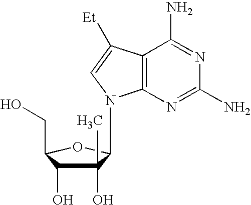

without protection of the free 2′- and/or 5′-OH.

In yet another particularly preferred sub-embodiment of the invention, the 2′-C-methyl branched nucleoside to be selectively esterified at the 3′-position is

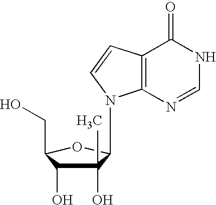

without protection of the free 2′- and/or 5′-OH.

In yet another particularly preferred sub-embodiment of the invention, the 2′-C-methyl branched nucleoside to be selectively esterified at the 3′-position is

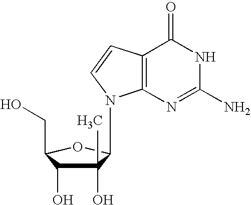

without protection of the free 2′- and/or 5′-OH.

In yet another particularly preferred sub-embodiment of the invention, the 2′-C-methyl branched nucleoside to be selectively esterified at the 3′-position is

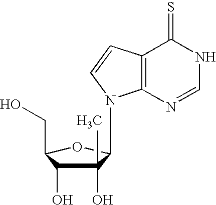

without protection of the free 2′- and/or 5′-OH.

In yet another particularly preferred sub-embodiment of the invention, the 2′-C-methyl branched nucleoside to be selectively esterified at the 3′-position is

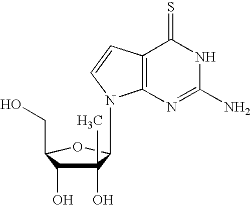

without protection of the free 2′- and/or 5′-OH.

In yet another particularly preferred sub-embodiment of the invention, the 2′-C-methyl branched nucleoside to be selectively esterified at the 3′-position is

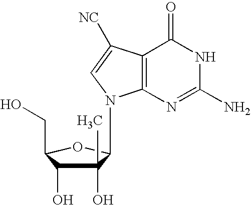

without protection of the free 2′- and/or 5′-OH.

In yet another particularly preferred sub-embodiment of the invention, the 2′-C-methyl branched nucleoside to be selectively esterified at the 3′-position is

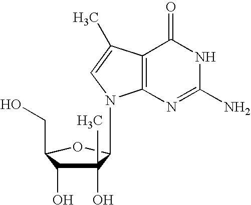

without protection of the free 2′- and/or 5′-OH.

In yet another particularly preferred sub-embodiment of the invention, the 2′-C-methyl branched nucleoside to be selectively esterified at the 3′-position is

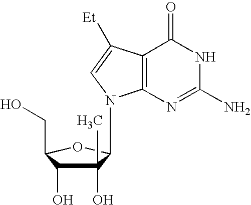

without protection of the free 2′- and/or 5′-OH.

In yet another particularly preferred sub-embodiment of the invention, the 2′-C-methyl branched nucleoside to be selectively esterified at the 3′-position is

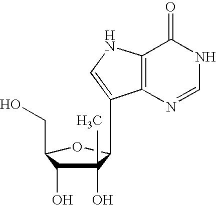

without protection of the free 2′- and/or 5′-OH.

In yet another particularly preferred sub-embodiment of the invention, the 2′-C-methyl branched nucleoside to be selectively esterified at the 3′-position is

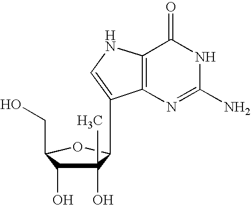

without protection of the free 2′- and/or 5′-OH.

In yet another particularly preferred sub-embodiment of the invention, the 2′-C-methyl branched nucleoside to be selectively esterified at the 3′-position is

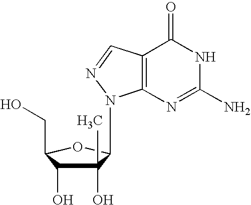

without protection of the free 2′- and/or 5′-OH.

In yet another particularly preferred sub-embodiment of the invention, the 2′-C-methyl branched nucleoside to be selectively esterified at the 3′-position is

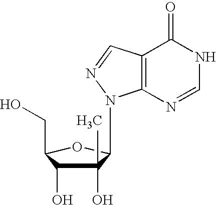

without protection of the free 2′- and/or 5′-OH.

Definitions and Alternative Reagents

The term “protected”, as used herein and unless specified otherwise, refers to a group that is added to an oxygen, nitrogen or phosphorus atom to prevent its further reaction or for other purposes. A wide variety of oxygen, nitrogen and phosphorus protecting groups are known to those skilled in the art of organic synthesis.

Examples of suitable protecting groups include, but not limited to, benzoyl; substituted or unsubstituted alkyl groups, substituted or unsubstituted aryl groups, substituted or unsubstituted silyl groups; substituted or unsubstituted aromatic or aliphatic esters, such as, for example, aromatic groups like benzoyl, toluoyls (e.g. p-toluoyl), nitrobenzoyl, chlorobenzoyl; ether groups such as, for example, —C—O-aralkyl, —C—O-alkyl, or —C—O-aryl; and aliphatic groups like acyl or acetyl groups, including any substituted or unsubstituted aromatic or aliphatic acyl, —(C═O)-aralkyl, —(C═O)-alkyl, or —(C═O)-aryl; wherein the aromatic or aliphatic moiety of the acyl group can be straight-chained or branched; all of which may be further optionally substituted by groups not affected by the reactions comprising the improved synthesis (see Greene et al., Protective Groups in Organic Synthesis, John Wiley and Sons, 2^{nd} Edition (1991)). For the use of ethers as protective groups, attention is directed to U.S. Pat. No. 6,229,008 to Saischek et al., herein incorporated by reference, wherein it is reported that the use of an ether as a protective group may offer significant advantages, particularly at the 5′-position of a pentofuranoside, for stability toward reagents and process conditions. This affords an ultimate advantage for separation, isolation, and purification of the desired product and thus, on the product's percent yield.

The amino acid protecting groups are preferably BOC (butoxycarbonyl), —(C═O)-aralkyl, —(C═O)-alkyl or —(C═O)-aryl. In one embodiment of the invention, the amino acid protecting group is BOC (butoxycarbonyl).

Throughout this application, the term “substituted” means single or multiple degrees of substitution by one or more named substituents. Where a single substituent is disclosed or claimed, the compound can be substituted once or more than once by that substituent. Where multiple substituents are disclosed or claimed, the substituted compound can be substituted independently by one or more of the disclosed or claimed substituent moieties, singly or plurally.

The term “alkyl”, as used herein and unless specified otherwise, refers to a saturated, straight, branched, or cyclic, primary, secondary or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, methylpentyl and dimethylbutyl. The term includes both substituted and unsubstituted alkyl groups. Moieties with which the alkyl group can be substituted in one or more positions are selected from the group consisting of halo (including fluorine, chlorine, bromine or iodine), hydroxyl (eg. CH₂OH), amino (eg., CH₂NH₂, CH₂NHCH₃ or CH₂N(CH₃)₂), alkylamino, arylamino, alkoxy, aryloxy, nitro, azido (eg., CH₂N₃), cyano (CH₂CN), sulfonic acid, sulfate, phosphonic acid, phosphate or phosphonate, any or all of which may be unprotected or further protected as necessary, as known to those skilled in the art and as taught, for example, in Greene et al., Protective Groups in Organic Synthesis, John Wiley and Sons, 2^{nd} Edition (1991).

The terms “alkylamino” and “arylamino” refer to an amino group that has one or more alkyl or aryl substituents, respectively.

The terms “alkaryl” and “alkylaryl” refer to an alkyl group with an aryl substituent. The terms “aralkyl” and “arylalkyl” refer to an aryl group with an alkyl substituent.

The term “halo” includes chloro, bromo, iodo, and fluoro.

The term “aryl”, as used herein, and unless specified otherwise, refers to phenyl, biphenyl or naphthyl. The term includes both substituted and unsubstituted moieties. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, any or all of which may be unprotected or further protected as necessary, as known to those skilled in the art and as taught, for example, in Greene et al., Protective Groups in Organic Synthesis, John Wiley and Sons, 2^{nd} Edition (1991).

The term “acyl” includes among other embodiments a carboxylic acid ester in which the non-carbonyl moiety of the ester group in one embodiment is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono-, di- or tri-phosphate ester, trityl or monomethoxytrityl, substituted benzyl, trialkylsilyl such as, for example, dimethyl-t-butylsilyl), or diphenylmethylsilyl. The terms “carboxylic acid” and “carboxylic acid ester” include the structures RC(═O)OH and RC(═O)O—R′, respectively. Here the non-carbonyl moiety, whether R or R′, is for example, straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy. Also intending for inclusion here are sulfonate esters such as alkyl or aralkyl sulphonyl including methanesulfonyl, the mono-, di- or tri-phosphate ester, trityl or monomethoxytrityl, substituted benzyl, trialkylsilyl such as, for example, dimethyl-t-butylsilyl), or diphenylmethylsilyl.

The term amino acid includes naturally occurring and synthetic α, β, γ, or δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In a preferred embodiment, the amino acid is in the L-configuration. In another preferred embodiment, the amino acid is L-valinyl. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, -βglycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl.

The term “non-natural amino acid” refers to a carboxylic acid having an amino group terminus but that is not found in nature. The term is intended to embrace both D- and L-amino acids, and any tautomeric or stereoisomeric forms thereof.

The term nucleoside base, includes but is not limited to purine or pyrimidine bases. Examples of purine or pyrimidine base include, but are not limited to, adenine, N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N-thioalkyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 2,6-diaminopurine, and 6-chloropurine. Functional oxygen and nitrogen groups on the base can be protected as necessary or desired. Suitable protecting groups are well known to those skilled in the art, and include trimethylsilyl, dimethylhexylsilyl, t-butyldimethylsilyl and t-butyldiphenylsilyl, trityl, alkyl groups, and acyl groups such as acetyl and propionyl, methanesulfonyl, and p-toluenesulfonyl. Alternatively, the purine or pyrimidine base can optionally substituted such that it forms a viable prodrug, which can be cleaved in vivo. Examples of appropriate substituents include acyl moiety, an amine or cyclopropyl (e.g., 2-amino, 2,6-diamino or cyclopropyl guanosine).

The process of the present invention is not limited to the use of the nucleoside, protected amino acid ester, and reagents exemplified. Suitable alternative reagents for the present invention may be used in place of those given above. For example, TEA (triethylamine) may be replaced by diisopropylethylamine, N-ethylmorpholine, or any tertiary aliphatic amine; DMF (dimethyl formamide) may be replaced by any polar solvent such as, for example, DMSO (dimethyl sulfoxide), although DMF is preferred based upon ease of handling and removability from the reaction mix; and CDI may be replaced by any reagent that enables coupling including, but not limited to, Mitsunobu reagents (e.g., diisopropyl azodicarboxylate and diethyl azodicarboxylate) with triphenylphosphine or carbodiimides other than carbonyl diimidazole.

The process of the present invention is not limited to the use of BOC as a protecting group. Other protecting groups such as, for example, substituted or unsubstituted silyl groups; substituted or unsubstituted ether groups like C—O-aralkyl, C—O-alkyl, or C—O-aryl; aliphatic groups such as acyl or acetyl groups having an alkyl moiety that is straight-chained or branched; and any such groups that would not adversely affect the materials, reagents and conditions of the present invention as known to those of skill in the art and as taught by Greene et al., Protective Groups in Organic Synthesis, John Wiley and Sons, 2^{nd} Edition (1991), may be used.

The process of the present invention is not limited to the use of the nucleoside, protected amino acid ester, and reagents exemplified. Suitable alternative reagents for the present invention may be used in place of those given above. For example, TEA (triethylamine) may be replaced by any other suitable amine, including but not limited to diisopropylethylamine, N-ethylmorpholine, or any tertiary aliphatic amine; DME (1,2-dimethoxyethane) may be replaced by any suitable polar aprotic solvent, such as THF (tetrahydrofuran) or any ether. Washes of the product slurry with THF just before and after the addition of MgSO₄ may be replaced by washes in acetone. Indeed, for scaled-up procedures, acetone is the preferred solvent.

In addition, DMF (dimethyl formamide) may be replaced by any polar solvent such as, for example, DMSO (dimethyl sulfoxide), although DMF is a preferred solvent based upon ease of handling and removability from the reaction mix.

EDC (1-[3-(dimethylamino)propyl]-3-ethyl-carbodiimide hydrochloride); also referred to as DEC) may be replaced by any reagent that enables coupling including, but not limited to, CDI (carbonyl diimidazole), BOP reagent (benzotriazol-1-yloxy-tris(dimethylamino)-phosphonium hexafluorophosphate), or similar coupling reagents as known to those skilled in the art.

Any organic solvents such as, for example, toluene may replace acetonitrile. Ammonia is an alternative reagent for use in place of sodium methoxide in methanol, and any polar solvent such as DMSO may replace DMF. Any number of other silylating reagents may replace TBDPSCl, any fluoride salt can replace NH₄F, and other acids such as TFA may be used to replace HCl.

The essential advantages of the present invention are its ability to be performed as a single step. Other advantages include the use of inexpensive reagents, and the requirement of only ordinary methods and equipment well known to those skilled in the art rather than complicated steps and expensive apparatus.

This invention is further illustrated in the following non-limiting examples. The working examples contained herein are set forth to aid in understanding the invention. They are illustrative of the process(es) and product(s) of the invention, but are not intended to and should not be interpreted to in any way limit the invention set forth in the claims that follow thereafter. Equivalent, similar or suitable solvents, reagents, or reaction conditions may be substituted for those particular solvents, reagents, and/or reaction conditions described herein without departing from the spirit and scope of the invention.

### EXAMPLES

### Example 1

2-Tert-butoxycarbonylamino-3-methyl-butyric acid 5-(4-amino--2-oxo-2H-pyrimidin-1-yl)-4-hydroxy-2-hydroxymethyl-4-methyl-tetrahydro-furan-3-yl ester

A solution of N-(tert-butoxycarbonyl)-L-valine (46.50 g, 214 mmol.), carbonyldiimidazole (34.70 g, 214 mmol.), and anhydrous tetrahydrofuran (1000 mL) in a 2 L round bottom flask, was stirred at 25° C. under argon for 1.5 hours and then at 40-50° C. for 20 minutes. In a separate 5 L 5-necked round bottom flask, equipped with an overhead stirrer, cooling tower, temperature probe, addition funnel, and an argon line was added 4-amino-1-(3,4-dihydroxy-5-hydroxymethyl-3-methyl-tetrahydro-furan-2-yl)-1H-pyrimidine-2-one (50.0 g, 195 mmol.) and anhydrous N,N-dimethylformamide (1000 mL). This mixture was heated at 100° C. for 20 minutes until all of the pyrimidine-2-one derivative compound went into solution, and then triethylamine (500 mL) and 4-dimethylaminopyridine (2.38 g, 19 mmol) were added to the solution. The mixture was next heated at 97° C. for 20 minutes and the tetrahydrofuran solution was added slowly through an addition funnel over a period of 2 hours, maintaining the temperature no lower than 82° C. The reaction mixture was heated at 82° C. for 1 hour and monitored by HPLC (product=68%, SM=11%, and impurity at about 12 min=17%, excluding dimethylaminopyridine). The reaction mixture was cooled to room temperature and then triethylamine and tetrahydrofuran were removed under vacuum at 30° C. The solution was then neutralized with acetic acid to a pH of 7.69. N,N-dimethylformamidine was removed under vacuum at 35° C. and chased with ethyl acetate (2×200 mL). The crude product was stirred with ethyl acetate (500 mL) and water (300 mL). The two layers were separated and the aqueous layer was extracted with ethyl acetate (500 mL). The combined organic layers were washed with an aqueous saturated brine solution (500 mL). Next the organic layer was extracted with an aqueous solution of malonic acid (4×400 mL, 10 wt. %). The organic layer was checked by TLC (silica, 20% methanol in dichloromethane) to make sure that all the desired product was removed from the organic layer. The acidic aqueous extracts were combined and cooled in an ice bath and neutralized with triethylamine to a pH of 7.40 so that the solids fell out of solution. Ethyl acetate then was added to the aqueous layer. The white solids were collected by vacuum filtration. Drying the obtained solids in vacuum gave 81.08 g of 99.01 pure (HPLC).

### Example 2

9-(2′-C-Methyl-3′-O-valinoyl β-D-ribofuranosyl)-6-N-methyl-adenine dihydrochloride

A solution of N-(tert-butoxycarbonyl)-L-valine (8.84 g, 41 mmol), carbonyl-diimidazole (6.60 g, 41 mmol) in tetrahydrofuran (200 mL) was stirred at room temperature under argon for one hour and then at 50° C. for 30 minutes. In a separate flask, equipped with an overhead stirrer, cooling tower, temperature probe, addition funnel, and an argon line, 9-(2′-C-methyl-β-D-ribofuranosyl)-6-N-methyl-adenine (1, FIG. 2, 10 g, 34 mmol) was dissolved in N,N-dimethylformamide (200 mL). This solution was heated to 100° C., triethylamine (100 mL) was added, and the temperature stabilized at 96° C. The activated Boc-valine solution was added quickly (over a 2 minute period) and the temperature was decreased to 81° C., then was stabilized at 85° C. The reaction mixture was stirred at that temperature and then cooled to 25° C. Triethylamine and tetrahydrofuran were removed under reduced pressure at 43° C. The solution then was cooled to 10° C. and neutralized with acetic acid to a pH of 7.7. Next, the mixture was diluted with methylene chloride (100 mL) and brine (100 mL). This mixture was agitated for 10 minutes, the layers were split, and the aqueous layer was back extracted with 2×100 mL of methylene chloride. The organic layer was extracted with a solution of 10% malonic acid in water (4×100 mL). Tert-butyl methyl ether (MTBE, 200 mL) was added to the combined malonic acid extracts, the mixture was cooled to 10° C., and triethylamine was added to achieve a pH of 7.1. The layers were separated and the aqueous layer was extracted with MTBE (2×200 mL). The combined MTBE layers were dried over anhydrous sodium sulphate and concentrated under vacuum to give a yellowish white solid. Drying the obtained solid in vacuum gavel 4.64 g (88% yield) of 97.87% pure (HPLC AUC) Boc-val nucleoside (2, FIG. 2).

A solution of compound 2 (13.0 g, 26.3 mmol) in ethanol (130 mL) was stirred in a round-bottomed flask equipped with an argon line and cooling tower. To this solution was added concentrated hydrochloric acid (37%, 6.5 mL). The reaction temperature was heated at reflux. Solid formation started after one hour of introducing the hydrochloric acid. After 3 hours, HPLC showed only 0.6% of starting material. Solids were then collected by vacuum filtration and the filter cake washed with ethanol (80 mL) and MTBE (40 mL). The crude product then was triturated with MTBE (100 mL) at 40° C. After drying the product under vacuum for 3 hours, 8.50 g (70%) of product (3, FIG. 2) was obtained in 98.55% purity (HPLC, AUC).

¹H NMR (DMSO-d₆) δ ppm 9.7 (broad s, 1H), 8.9-8.8 (m, 4H,), 8.45 (s, 1H), 6.04 (s, 1H, H-1′), 5.43 (d, 1H, H-3′, J=5.1 Hz), 4.30-4.28 (m, 1H, H-4′), 3.96-3.95 (m, 1H, CH), 3.85-3.64 (m, 2H, H-5′, H-5″), 3.10 (d, 3H, CH₃NH, J=2.1 Hz), 2.3-2.2 (m, 1H, CH), 1.02-0.97 (m, 6H, (CH₃)₂CH), 0.92 (s, 3H, CH₃). ¹³C NMR (DMSO-d₆) δ ppm 167.99, 150.26, 146.58, 140.67, 118.99, 91.32, 80.61, 78.89, 74, 56, 29.29, 29.0, 25.50, 20.48, 18.55, 17.72.

This invention has been described with reference to its preferred embodiments. Variations and modifications of the invention will be obvious to those skilled in the art from the foregoing detailed description of the invention.

We claim:

1. A process for selectively esterifying the 3′ hydroxyl position of a 2′-branched ribofuranosyl nucleoside, optionally in a one pot system, comprising reacting:
a) a 2′ branched ribofuranosyl nucleoside,
b) an optionally protected organic acid;
c) a coupling reagent; and
d) base, optionally in the presence of a base catalyst.

2. The process of claim 1, wherein the 2′ branched ribofuranosyl nucleoside is a 2′-C-methyl branched nucleoside of the formula: 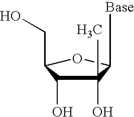
wherein:
Base is a purine, pyrimidine, pyrrolopyrimidine, triazolopyridine, imidazolopyridine, or a pyrazolopyrimidine.

3. The process of claim 2, wherein the Base is a pyrimidine base.

4. The process of claim 2, wherein the pyrimidine base is selected from the group consisting of thymine, cytosine, 5-fluorocytosine, 5-methylcytosine, 6-aza-pyrimidine, including 6-azacytosine, 2- and/or 4-mercaptopyrmidine, uracil, 5-halouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, and C⁵-aminopyrimidine.

5. The process of claim 3, wherein the pyrimidine base is selected from the group consisting of

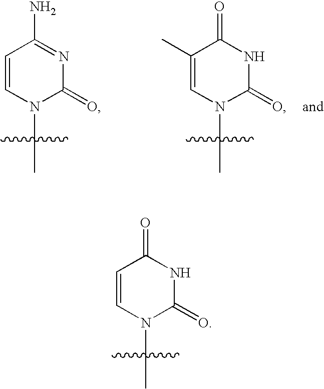

6. The process of claim 2, wherein the Base is a purine base.

7. The process of claim 6, wherein the purine base is selected from the group consisting of N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-thioalkyl purine, N²-alkylpurines, N²-alkyl-6-thiopurines, N²-alkylpurines, N²-alkyl-6-thiopurines, 5-azacytidinyl, guanine, adenine, hypoxanthine, 2,6-diaminopurine, and 6-chloropurine.

8. The process of claim 6, wherein the purine base is selected from the group consisting of

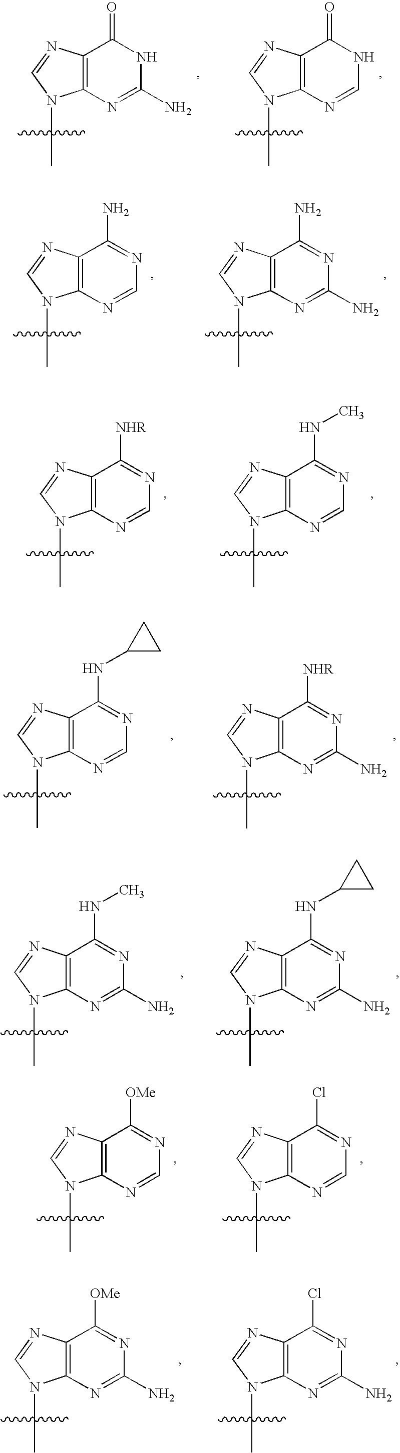

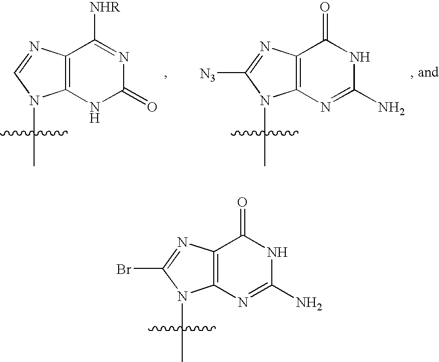

9. The process of claim 16, wherein the Base is a pyrrolopyrimidine.

10. The process of claim 16, wherein the Base is a triazolopyridine, an imidazolopyridine, or a pyrazolopyrimidine.

11. The process of claim 1, wherein the 2′-C-methyl branched nucleoside to be selectively esterified at the 3′-position is 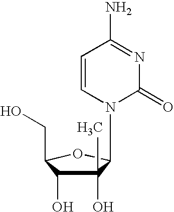
wherein the reaction optionally occurs without protection of the free 2′- and/or 5′-OH.

12. The process of claim 1, wherein the 2′-C-methyl branched nucleoside to be selectively esterified at the 3′-position is 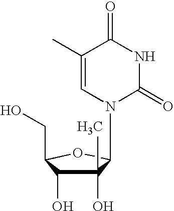
wherein the reaction optionally occurs without protection of the free 2′- and/or 5′-OH.

13. The process of claim 1, wherein the 2′-C-methyl branched nucleoside to be selectively esterified at the 3′-position is 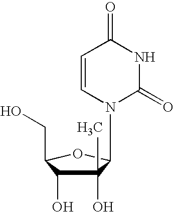
wherein the reaction optionally occurs without protection of the free 2′- and/or 5′-OH.

14. The process of claim 1, wherein the 2′-C-methyl branched nucleoside to be selectively esterified at the 3′-position is 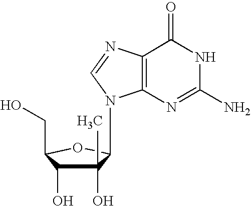
wherein the reaction optionally occurs without protection of the free 2′- and/or 5′-OH.

15. The process of claim 1, wherein the 2′-C-methyl branched nucleoside to be selectively esterified at the 3′-position is 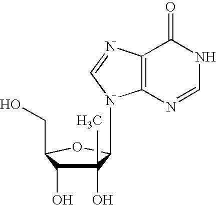
wherein the reaction optionally occurs without protection of the free 2′- and/or 5′-OH.

16. The process of claim 1, wherein the 2′-C-methyl branched nucleoside to be selectively esterified at the 3′-position is 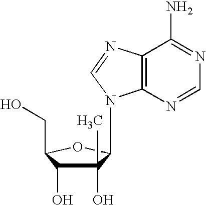
wherein the reaction optionally occurs without protection of the free 2′- and/or 5′-OH.

17. The process of claim 1, wherein the 2′-C-methyl branched nucleoside to be selectively esterified at the 3′-position is 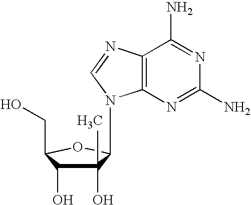
and wherein the reaction optionally occurs without protection of the free 2′- and/or 5′-OH.

18. The process of claim 1, wherein the 2′-C-methyl branched nucleoside to be selectively esterified at the 3′-position is 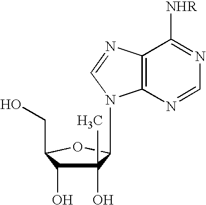
wherein the reaction optionally occurs without protection of the free 2′- and/or 5′-OH; and
wherein R is methyl, ethyl, propyl, isopropyl, cyclopropyl, butyl, isobutyl, t-butyl, pentyl, cyclopentyl, isopentyl, or neopentyl.

19. The process of claim 1, wherein the 2′-C-methyl branched nucleoside to be selectively esterified at the 3′-position is 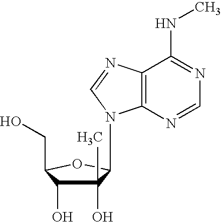
wherein the reaction occurs optionally without protection of the free 2′- and/or 5′-OH.

20. The process of claim 1, wherein the 2′-C-methyl branched nucleoside to be selectively esterified at the 3′-position is 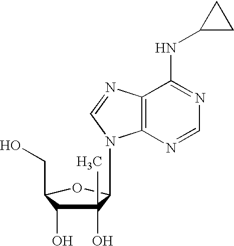
and wherein the reaction occurs optionally without protection of the free 2′- and/or 5′-OH.

21. The process of claim 1, wherein the 2′-C-methyl branched nucleoside to be selectively esterified at the 3′-position is 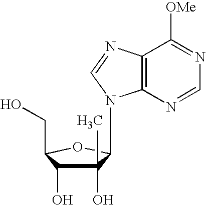
wherein the reaction occurs optionally without protection of the free 2′- and/or 5′-OH.

22. The process of claim 1, wherein the 2′-C-methyl branched nucleoside to be selectively esterified at the 3′-position is 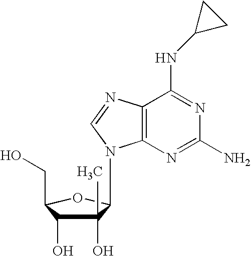
and wherein the reaction occurs optionally without protection of the free 2′- and/or 5′-50H.

23. The process of claim 1 wherein the 2′-C-methyl branched nucleoside to be selectively esterified at the 3′-position is 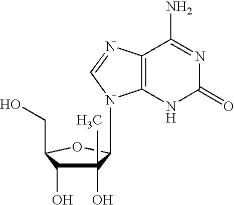
wherein the reaction occurs optionally without protection of the free 2′- and/or 5′-OH.

24. The process of claim 1, wherein the 2′-C-methyl branched nucleoside to be selectively esterified at the 3′-position is 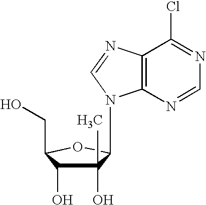
wherein the reaction occurs optionally without protection of the free 2′- and/or 5′-OH.

25. The process of claim 1, wherein the 2′-C-methyl branched nucleoside to be selectively esterified at the 3′-position is 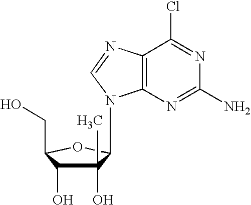
wherein the reaction occurs optionally without protection of the free 2′- and/or 5′-OH.

26. The process of claim 1, wherein the 2′-C-methyl branched nucleoside to be selectively esterified at the 3′-position is 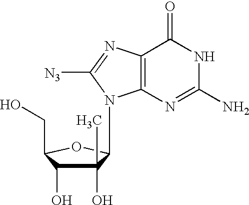
wherein the reaction occurs optionally without protection of the free 2′- and/or 5′-OH.

27. The process of claim 1, wherein the 2′-C-methyl branched nucleoside to be selectively esterified at the 3′-position is 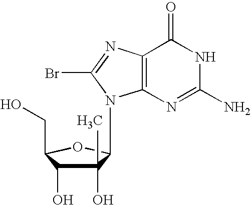
wherein the reaction optionally occurs without protection of the free 2′- and/or 5′-OH.

28. The process of claim 1, wherein the 2′-C-methyl branched nucleoside to be selectively esterified at the 3′-position is 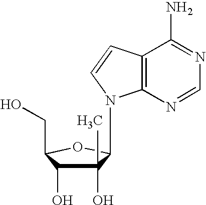
wherein the reaction optionally occurs without protection of the free 2′- and/or 5′-OH.

29. The process of claim 1, wherein the 2′-C-methyl branched nucleoside to be selectively esterified at the 3′-position is 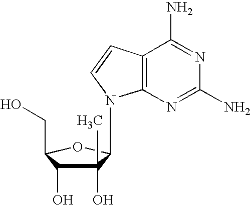
wherein the reaction optionally occurs without protection of the free 2′- and/or 5′-OH.

30. The process of claim 1, wherein the 2′-C-methyl branched nucleoside to be selectively esterified at the 3′-position is 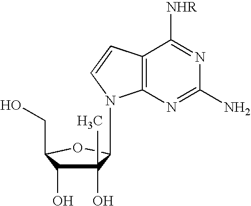
wherein the reaction optionally occurs without protection of the free 2′- and/or 5′-OH.
wherein R is methyl, ethyl, propyl, isopropyl, cyclopropyl, butyl, isobutyl, t-butyl, pentyl, cyclopentyl, isopentyl, or neopentyl.

31. The process of claim 1, wherein the 2′-C-methyl branched nucleoside to be selectively esterified at the 3′-position is 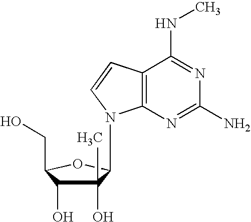
wherein the reaction optionally occurs without protection of the free 2′- and/or 5′-OH.

32. The process of claim 1, wherein the 2′-C-methyl branched nucleoside to be selectively esterified at the 3′-position is 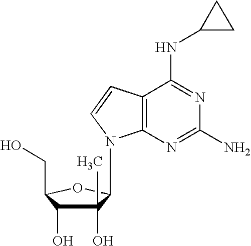
wherein the reaction optionally occurs without protection of the free 2′- and/or 5′-OH.

33. The process of claim 1, wherein the 2′-C-methyl branched nucleoside to be selectively esterified at the 3′-position is 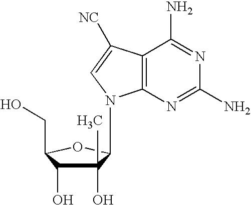
wherein the reaction optionally occurs without protection of the free 2′- and/or 5′-OH.

34. The process of claim 1, wherein the 2′-C-methyl branched nucleoside to be selectively esterified at the 3′-position is 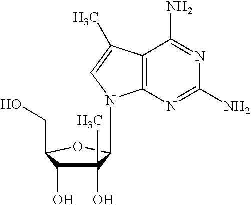
wherein the reaction optionally occurs without protection of the free 2′- and/or 5′-OH.

35. The process of claim 1, wherein the 2′-C-methyl branched nucleoside to be selectively esterified at the 3′-position is 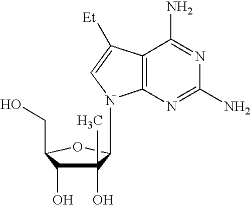
wherein the reaction optionally occurs without protection of the free 2′- and/or 5′-OH.

36. The process of claim 1, wherein the 2′-C-methyl branched nucleoside to be selectively esterified at the 3′-position is 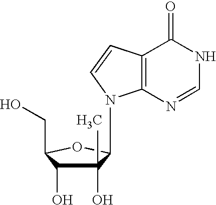
wherein the reaction optionally occurs without protection of the free 2′- and/or 5′-OH.

37. The process of claim 1, wherein the 2′-C-methyl branched nucleoside to be selectively esterified at the 3′-position is 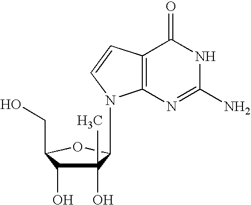
wherein the reaction optionally occurs without protection of the free 2′- and/or 5′-OH.

38. The process of claim 1, wherein the 2′-C-methyl branched nucleoside to be selectively esterified at the 3′-position is 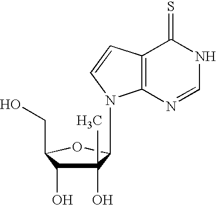
wherein the reaction optionally occurs without protection of the free 2′- and/or 5′-OH.

39. The process of claim 1, wherein the 2′-C-methyl branched nucleoside to be selectively esterified at the 3′-position is 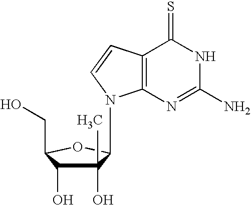
wherein the reaction optionally occurs without protection of the free 2′- and/or 5′-OH.

40. The process of claim 1, wherein the 2′-C-methyl branched nucleoside to be selectively esterified at the 3′-position is 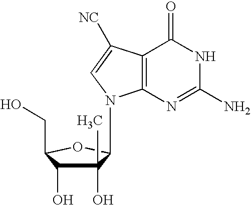
wherein the reaction optionally occurs without protection of the free 2′- and/or 5′-OH.

41. The process of claim 1, wherein the 2′-C-methyl branched nucleoside to be selectively esterified at the 3′-position is 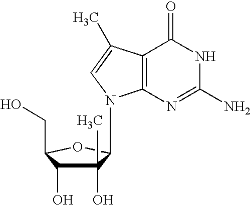
wherein the reaction optionally occurs without protection of the free 2′- and/or 5′-OH.

42. The process of claim 1, wherein the 2′-C-methyl branched nucleoside to be selectively esterified at the 3′-position is 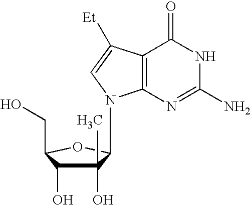
wherein the reaction optionally occurs without protection of the free 2′- and/or 5′-OH.

43. The process of claim 1, wherein the 2′-C-methyl branched nucleoside to be selectively esterified at the 3′-position is 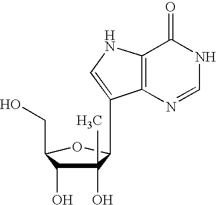
wherein the reaction optionally occurs without protection of the free 2′- and/or 5′-OH.

44. The process of claim 1, wherein the 2′-C-methyl branched nucleoside to be selectively esterified at the 3′-position is 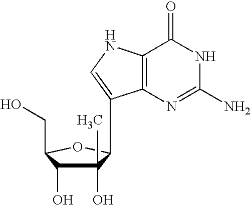
wherein the reaction optionally occurs without protection of the free 2′- and/or 5′-OH.

45. The process of claim 1, wherein the 2′-C-methyl branched nucleoside to be selectively esterified at the 3′-position is 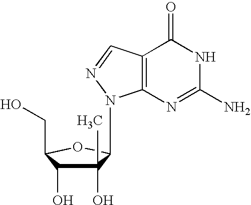
wherein the reaction optionally occurs without protection of the free 2′- and/or 5′-OH.

46. The process of claim 1, wherein the 2′-C-methyl branched nucleoside to be selectively esterified at the 3′-position is 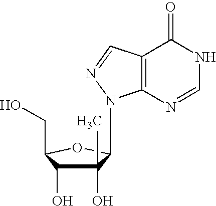
wherein the reaction optionally occurs without protection of the free 2′- and/or 5′-OH.

47. The process of claim 1, wherein the optionally protected organic acid is an optionally protected amino acid.

48. The process of claim 50, wherein the optionally protected amino acid is an optionally protected L-valinoyl.

49. The process of claim 51, wherein the optionally protected L-valinoyl is Boc-L-valinoyl.

50. The process of claim 1, wherein the coupling reagent is selected from the group consisting of EDC (1-[3-(dimethylamino)-propyl]-3-ethyl-carbodiimide hydrochloride); CDI (carbonyldiimidazole), BOP reagent (benzotriazol-1-yloxy-tris(dimethylamino)-phosphonium hexafluorophosphate), and Mitsunobu reagents with triphenylphosphine.

51. The process of claim 1, wherein the coupling reagent is a carbodiimide.

52. The process of claim 51, wherein the coupling reagent is CDI.

53. The process of claim 1, wherein the base is selected from the group consisting of TEA (triethylamine), diisopropylethylamine, and N-ethylmorpholine.

54. The process of claim 1, wherein the base is a tertiary amine.

55. The process of claim 54, wherein the tertiary amine is triethylamine.

56. The process of claim 1, wherein the base catalyst is DMAP.

57. The process of claim 1, wherein the molar ratio of the optionally protected organic acid and the nucleoside is 1.0 to 1.5.

58. The process of claim 57, wherein the molar ratio 1.0 to about 1.2.

59. The process of claim 1, wherein the molar ratio of the coupling agent and the nucleoside is 1.0 to 1.5.

60. The process of claim 59, wherein the molar ratio is 1.0 to 1.2.

61. The process of claim 1, wherein the reaction is conducted at a temperature of at least 80° C. for at least 20 minutes.

62. The process of claim 61, wherein the reaction occurs under argon gas.

63. The process of claim 1, wherein the 2′-branched ribofuranosyl nucleoside is solubilized in a solvent.

64. A process for selectively esterifying the 3′ hydroxyl position of a 2′-branched ribofuranosyl nucleoside comprising:
a) heating a first solution of a 2′ branched ribofuranosyl nucleoside in an organic solvent at temperature and for a time sufficient to dissolve the nucleoside;
b) adding a tertiary amine and a base catalyst to the first solution; and
c) adding a second solution, comprising a protected amino acid and a carbodiimide coupling reagent in an organic solvent, to the first solution.

65. The process of claim 64 wherein in step a) the first solution is heated to at least 80° C. for at least 20 minutes.

66. The process of claim 64 wherein in step c) the first solution is maintained at a temperature of at least 80° C., and the second solution is added over a time period of at least one hour.

67. The process of claim 66, further comprising heating the combined first and second solutions at a temperature of at least 80° C. for at least about one half hour.

68. The process of claim 64, wherein the organic solvent in the first solution is DMF.

69. The process of claim 64, wherein the organic solvent in the second solution is THF or DMF.

70. The process of claim 64, further comprising neutralizing the product solution with an acid.

71. The process of claim 64, wherein the tertiary amine is triethylamine and the base catalyst is DMAP.

72. The process of claim 64, wherein the protected amino acid is a protected L-valinoyl amino acid.

73. The process of claim 1, wherein the reaction occurs in a solvent or mixture of solvents selected from the group consisting of polar aprotic solvent.

74. The process of claim 73, wherein the solvent is selected from the group consisting of acetone, ethyl acetate, dithianes, THF, dioxane, acetonitrile, dichloromethane, dichloroethane, diethyl ether, pyridine, dimethylformamide (DMF), DME, dimethylsulfoxide (DMSO), dimethylacetamide, and combination thereof.
